(19) United States
(12) Patent Application Publication
(10) Pub. No.: US 2022/0226286 A1
(43) Pub. Date: Jul. 21, 2022

(54) COMPOSITIONS AND METHODS FOR THE TREATMENT AND MITIGATION OF ALCOHOL-INDUCED SKIN FLUSHING

(71) Applicant: EKYJ Consulting II , LLC, Las Vegas, NV (US)

(72) Inventor: Edward K. Y. Jung, Bellevue, WA (US)

(73) Assignee: EKYJ Consulting II, LLC, Las Vegas, NV (US)

(21) Appl. No.: 17/613,680

(22) PCT Filed: May 22, 2020
(86) PCT No.: PCT/US2020/034273

§ 371 Date: Nov. 23, 2021

Related U.S. Application Data
(60) Provisional application No. 62/852,000, filed on May 23, 2019.

(51) Int. Cl. A61K 31/415; A61K 31/375; A61K 45/06; A61K 38/06; A61K 33/26; A61K 33/30; A61K 33/24

(52) U.S. Cl. CPC A61K 31/415; A61K 31/375; A61K 45/06; A61K 33/24; A61K 33/26; A61K 33/30; A61K 38/063

(57) ABSTRACT

The present application relates to pharmaceutical compositions for use in the prevention and treatment of alcohol-induced hypersensitivity reactions including alcohol-induced skin flushing.

### CROSS-REFERENCE TO RELATED APPLICATIONS

This application claims priority from U. S. Provisional Application No. 62/852,000 filed May 23, 2019, which is hereby incorporated herein by reference in its entirety.

### SUMMARY

The present application relates to pharmaceutical compositions comprising a therapeutically effective amount of 4-methylpyrazole, or pharmaceutically acceptable salts, hydrates, polymorphs, or solvates thereof, and a pharmaceutically acceptable carrier; wherein the pharmaceutical composition is configured for oral administration; and wherein the pharmaceutical composition further comprises at least one pharmaceutically-inert coating useful in masking the odor of 4-methylpyrazole. In some embodiments, the at least one pharmaceutically-inert coating is selected from group consisting of a hydroxyalkyl cellulose, an anti-tackiness agent, a plasticizer; a sugar, a methacrylate copolymer, a hydroxyalkyl cellulose, and a water soluble polymer. In some embodiments, the pharmaceutical composition is configured as an oral dosage form selected from a powder, tablet, lozenge, chewing gum, a pill, a capsule, a microcapsule, a caplet, an orally disintegrating tablet, an osmotic controlled-release oral delivery system, or any combination thereof. Some embodiments further comprise n-acetyl cysteine, ampelopsin, Withania somifera/ashwaganda, L-cystine, S-acetyl glutathione, molybdenum, iron, zinc, an iron chelator, L-ascorbic acid, L-threonine or any combination thereof. In some embodiments, the iron chelator comprises curcumin, quercetin, inositol hexakisphosphate (IP6), or any combination thereof. In some embodiments, the pharmaceutically acceptable carrier is water. In some embodiments, the pharmaceutical composition further comprises a taste masking agent, an odor masking agent, or a combination thereof. In some embodiments, the therapeutically effective amount of 4-methylpyrazole is an amount between about 10 and about 20 mg/kg. In some embodiments, the therapeutically effective amount of 4-methylpyrazole is an amount resulting in a plasma concentration of about 0.1 μmol/L.

Some embodiments are directed to pharmaceutical compositions comprising a therapeutically effective amount of 4-methylpyrazole or pharmaceutically acceptable salts, hydrates, polymorphs, or solvates thereof, and a pharmaceutically acceptable carrier; wherein the pharmaceutical composition is configured for transdermal administration. In some embodiments, the pharmaceutical composition further comprises n-acetyl cysteine, ampelopsin, Withania somifera/ashwaganda, L-cystine, S-acetyl glutathione, molybdenum, iron, zinc, an iron Chelator, L-ascorbic acid, L-threanine or any combination thereof. In some embodiments, the iron chelator comprises curcumin, quercetin, inositol hexakisphosphate, or any combination thereof.

Some embodiments are directed to methods of treating and/or preventing an alcohol-induced hypersensitivity reaction in a subject comprising administering a pharmaceutical composition disclosed herein. In some embodiments, the alcohol-induced hypersensitivity reaction is selected from flushing, elevated heart rate, palpitations, hypotension, nausea, dizziness, headache, vomiting, diarrhea, upset stomach, ataxia, confused consciousness, urticarial, systemic dermatitis, allergic rhinitis, bronchoconstriction, exacerbation of asthmatic bronchoconstriction, cardiovascular collapse, allergic conjunctivitis, atopic dermatitis, eosinophilic esophagitis, anaphylaxis, chronic bronchitis and chronic obstructive pulmonary disease (COPD) and any combination thereof. In some embodiments, alcohol flushing is characterized by facial redness, increased skin temperature, elevated heart rate, decreased diastolic blood pressure or any combination thereof. In some embodiments, the pharmaceutical composition is administered before the subject consumes alcohol. In some embodiments, the pharmaceutical composition is administered about sixty minutes to about fifteen minutes before the subject consumes ethanol. In some embodiments, the pharmaceutical composition is administered concurrently with the subject's consumption of ethanol or after the subject has consumed ethanol.

Some embodiments, are directed to methods of eliminating acetaldehyde formed from ethanol in a subject comprising administering a pharmaceutical composition as described herein.

Some embodiments, are directed to methods of reducing and/or eliminating acetaldehyde formed from ethanol in the oral cavity, esophagus, stomach, large intestine, or a combination thereof, in a subject, comprising administering a pharmaceutical composition as described herein.

Some embodiments, are directed to methods of reducing and/or eliminating acetaldehyde blood levels in a subject, comprising administering a pharmaceutical composition as described herein.

Some embodiments, are directed to methods of reducing and/or eliminating acetaldehyde from the digestive tract in a subject comprising administering a pharmaceutical composition as described herein.

Some embodiments, are directed to methods of inhibiting mitochondrial aldehyde dehydrogenase 2 (ALDH2) in a subject in a subject comprising administering a pharmaceutical composition as described herein.

Some embodiments, are directed to methods for reducing a risk in a subject for a disease or disorder caused by consumption of ethanol in a subject comprising administering a pharmaceutical composition as described herein.

In some embodiments, the disease or disorder is selected from upper aerodigestive tract cancers, digestive tract cancers or breast cancer. In some embodiments, the upper aerodigestive tract cancer comprises esophageal, oropharynx, hypopharynx, larynx, head or neck cancer. In some embodiments, the digestive tract cancer comprises stomach or colon cancer. In some embodiments, the disease or disorder comprises late-onset Alzheimer's disease, hypertension, myocardial infarction, Parkinson's disease, amyotrophic lateral sclerosis, and cerebral ischemia.

Some embodiments, are directed to methods of blocking the histamine-releasing effect of acetaldehyde in a subject comprising administering a pharmaceutical composition as described herein.

Some embodiments, are directed to methods of blocking alcohol induced acetaldehyde production in a subject comprising administering a pharmaceutical composition as described herein. In some embodiments, the subject is heterozygous or homozygous for the aldehyde dehydrogenase 2 (ALDH2) allele termed Glu487lys, ALDH2*2, ALDH2*487lys, Glu487lys, or rs671.

Some embodiments further comprising testing the subject for the presence of aldehyde dehydrogenase 2 (ALDH2) allele termed Glu487lys, ALDH2*2, ALDH2*487lys, Glu504lys, or rs671. In some embodiments, testing the subject for the presence of aldehyde dehydrogenase 2 (ALDH2) allele termed Glu487lys, ALDH2*2, ALDH2*487lys, Glu504lys, or rs671 comprises obtaining a breath sample from the subject; measuring ethanol and acetaldehyde levels in the breath sample; and determining the ratio of acetaldehyde level-to-ethanol level in the breath sample wherein a ratio of acetaldehyde level-to-ethanol level of about 23.3 or higher is indicative of the presence of aldehyde dehydrogenase 2 (ALDH2) allele termed Glu487lys, ALDH2*2, ALDH2*487lys, Glu504lys, or rs671; and administering a pharmaceutical composition disclosed herein, to the subject if the subject has a ratio of acetaldehyde level-to-ethanol level of about 23.3 or higher. In some embodiments, measuring ethanol and acetaldehyde levels in the breath sample is done by semiconductor gas chromatography. In some embodiments, the breath sample is obtained following consumption of alcohol. In some embodiments, testing the subject for the presence of aldehyde dehydrogenase 2 (ALDH2) allele termed Glu487lys, ALDH2*2, ALDH2*487lys, Glu504lys, or rs671 comprises obtaining a biological sample from the subject; isolating genomic DNA from the biological sample; identifying the presence of the aldehyde dehydrogenase 2 (ALDH2) allele termed Glu487lys, ALDH2*2, ALDH2*487lys, Glu504lys, or rs671; and administering a pharmaceutical described herein to the subject if the aldehyde dehydrogenase 2 (ALDH2) allele termed Glu487lys, ALDH2*2, ALDH2*487lys, Glu504lys, or rs671 is present. In some embodiments, the biological sample is a swab sample. In some embodiments, the biological sample is a blood sample.

### DETAILED DESCRIPTION

The present application relates to pharmaceutical compositions for use in the prevention and treatment of alcohol-induced hypersensitivity reactions including alcohol-induced skin flushing.

Alcohol, also known by its chemical name as ethanol, is consumed for a variety of social, recreational, and medicinal purposes in humans As used herein the terms “alcohol” and “ethanol” are used interchangeably. Excessive alcohol consumption causes injury to a wide variety of tissues including liver, brain, skeletal, and cardiac muscle and is responsible for considerable public health morbidity and mortality. Many of these effects of alcohol are mediated by acetaldehyde, which is produced during alcohol metabolism in a two-step pathway in which alcohol is oxidized by alcohol dehydrogenase (ADH) to acetaldehyde, which is in turn quickly metabolized into acetic acid by aldehyde dehydrogenase (ALDH), a mitochondrial liver enzyme.

The ADH and ALDH genes display polymorphisms that modulate individual differences in alcohol-oxidizing capability (Bosron et al., Hepatology 1986, 6, 502-510). East Asian populations carry a variant allele of alcohol dehydrogenase subtype 2 (ADH2*2) that encodes an ADH enzyme with a R47H amino acid substitution (Matsuo et al., Carcinogenesis 2006, 27(5), 1018-1023; Tamakoshi et al., Alcohol 2003, 38, 407-410). The H47 ADH enzyme is “superactive,” exhibiting a Vmax about 40 times higher than the less active R47 ADH enzyme encoded by the “typical” allele (ADH2*1) (Bosron et al., Hepatology 1986, 6, 502-510; Yoshida ct al., Prog. Nucleic Acid Res. Mol. Biol. 1991, 40, 255-287). The ADH2*2 allele is associated with the accumulation of acetaldehyde (Crabb et al., Proc. Nutr. Soc. 2004, 63(1), 49-63).

Also prevalent in East Asian populations is a variant allele of aldehyde dehydrogenase subtype 2 (ALDH2*2, and also known as Glu487lys, ALDH2*487lys, Glu504lys, or rs671) that encodes for an ALDH enzyme with an E487K amino acid substitution (Chen et al., Am. J. Hum. Genet. 1999, 65(3), 795-807). The K487ALDH enzyme exhibits reduced activity that results in a 40%-90% reduction in the rate of acetaldehyde removal when compared to the more active E487 ALDH2 enzyme encoded by the “typical” allele (ALDH2*1), such that persons who express the variant allele display reduced or absent ALDH2 activity.

Acetaldehyde is linked to acute symptoms such as facial flushing, tachycardia, shortness of breath, dizziness, nausea, vomiting and headache, as well as to increased long-term health risks for cancers of the upper digestive tract, breast cancer, liver disease, Alzheimer's disease, hypertension and myocardial infarction (see Visapaa et al., Gut 2004, 53, 871-876; Yokoyama et al., Jpn. J. Clin. Oncol. 2003, 33(3), 111-121; Ohsawa et al., J. Hum. Genet. 2003, 48, 404-409; and references cited therein). People who express the ALDH2*2 allele having reduced or absent ALDH2 activity exhibit alcohol-related sensitivity, for example, facial flushing, tachycardia, etc., when drinking small portions of ethanol (Goedde et al., Hum. Genet. 1992, 88, 344-346; Xiao et al., J. Clin. Invest. 1995, 96, 2180-2186). Therefore, reducing acetaldehyde levels in people with reduced or absent ALDH2 activity may be helpful in reducing the acute systems and long-term health risks experienced by these people when they consume ethanol.

4-Methylpyrazole (also known as fomepizole or 4-MP) inhibits alcohol dehydrogenase (ADH), the enzyme that oxidizes alcohols as part of a two-step metabolic removal pathway in which ethanol is oxidized by ADH to acetaldehyde, which is in turn oxidized by aldehyde dehydrogenase (ALDH) to acetic acid.

In some embodiments, the compounds for use in the methods described herein may be formulated as pharmaceutical compositions. Pharmaceutical compositions of this invention may comprise the compounds described herein or a pharmaceutically acceptable salt thereof and a pharmaceutically acceptable carrier. Such compositions may optionally comprise at least one additional therapeutic agent useful for the prevention and treatment of alcohol-induced hypersensitivity reactions including alcohol-induced skin flushing.

The compounds of this invention may be employed in a conventional manner for controlling the disease described herein, including, but not limited to, alcohol-induced hypersensitivity reactions including alcohol-induced skin flushing, and for treating diseases or reducing the advancement or severity of effects. Such methods of treatment, their dosage levels and requirements may be selected by those of ordinary skill in the art from available methods and techniques. For example, the compounds of this invention may be combined with a pharmaceutically acceptable adjuvant for administration to a patient having, or being susceptible to alcohol-induced hypersensitivity reactions including alcohol-induced skin flushing in a pharmaceutically acceptable manner and in an amount effective to lessen the occurrence and severity of the condition.

In each of the embodiments disclosed herein, the compounds and methods can be utilized with or on a subject in need of such treatment, which can also be referred to as “in need thereof.” As used herein, the phrase “in need thereof” means that the subject has been identified as having a need for the particular method or treatment and that the treatment has been given to the subject for that particular purpose.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

As used herein, the terms “combination,” “combined,” and related terms refer to the simultaneous or sequential administration of therapeutic agents in accordance with this invention. For example, a described compound may be administered with another therapeutic agent simultaneously or sequentially in separate unit dosage forms or together in a single unit dosage form. Accordingly, the present invention provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

The term “pharmaceutically acceptable excipient” refers to a non-toxic excipient that may be administered to a patient, together with a compound of this invention, and which does not destroy the pharmacological activity thereof. Pharmaceutically acceptable excipients that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Pharmaceutically acceptable excipients that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as a-tocopherol, polyethyleneglycol 1000 succinate, or other similar polymeric delivery matrices.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology). In some embodiments, the therapeutically effective amount of a compound represents the daily dose a particular compound. In some embodiments, the daily dose of a particular compound may be administered as a single daily dose or may be divided into two or more doses of equal or unequal amounts administered throughout the day.

The compounds described herein can form acid addition salts thereof. It will be appreciated that for use in medicine the salts of the compounds described herein should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms. Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The compounds described herein may be prepared in crystalline or non-crystalline form, and, if crystalline, may optionally be solvated, eg. as the hydrate. This invention includes within its scope stoichiometric solvates (eg. hydrates) as well as compounds containing variable amounts of solvent (eg. water). Certain compounds described herein are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

Although any methods and materials similar or equivalent to those described herein can be used in the practice or testing of embodiments of the present invention, the preferred methods, devices, and materials are now described.

Alternatively, the compounds of this invention may be used in compositions and methods for treating or protecting individuals against the conditions described herein, including but not limited to a neurodegenerative disease, over extended periods of time. The compounds may be employed in such compositions either alone or together with other compounds of this invention in a manner consistent with the conventional utilization of such compounds in pharmaceutical compositions. For example, a compound of this invention may be combined with pharmaceutically acceptable adjuvants conventionally employed in vaccines and administered in prophylactically effective amounts to protect individuals over an extended period of time against the diseases described herein, including, but not limited to, neurodegenerative diseases.

The present application relates to pharmaceutical compositions comprising a therapeutically effective amount of 4-methylpyrazole, or pharmaceutically acceptable salts, hydrates, polymorphs, or solvates thereof, and a pharmaceutically acceptable carrier; wherein the pharmaceutical composition is configured for oral administration.

In some embodiments, the pharmaceutical composition further comprises at least one pharmaceutically-inert coating useful in masking the odor of 4-methylpyrazole. In yet other embodiments, the pharmaceutical compositions described herein can be encapsulated. In some embodiments, encapsulation mediums include, but are not limited to capsules, soft gel caps, gel caps, coatings, or any combination thereof. In some embodiments, coatings may include, but are not limited to, a film, a wax, a varnish, a glaze, a polymer coating, a sugar coating, a polysaccharide based coating, an enteric coating, or a combination thereof.

In some embodiments, the at least one pharmaceutically-inert coating is selected from group consisting of a hydroxyalkyl cellulose, hydroxypropyl cellulose, an anti-tackiness agent, a plasticizer; a sugar, a methacrylate copolymer, a hydroxyalkyl cellulose, and a water soluble polymer. Examples of ant-tackiness agents, include but are not limited to talc (Alphafil 500), silicon dioxide, silica hydrogel, microcrystalline cellulose, alkali stearates, starch, and combinations thereof. Examples of platiciziers include but are not limited to propylene glycol, glycerin, trimethylolpropane, polyethylene glycols, dibutyl sebacate, acetylated monoglycerides, diethylphthalate, triacetin, glyceryl triacetate, aceryltriethyl citrate, triethyl citrate and combinations thereof. Examples of water soluble polymers include but are not limited to hydroxypropyl cellulose, hydroxypropyl methylcellulose, acacia, sodium carboxymethylcellulose, dextrin, alginic acid, ethylcellulose resin, gelatin, guar gum, liquid glucose, methylcellulose, pregelatinized starch, sodium alginate, starch, zein, polyvinylpyrrolindone, vinylpyrrolidone-vinyl acetate copolymer, vinyl acetate-crotonic acid copolymer, ethyl acrylate-methacrylic acid copolymer, and combinations thereof.

In some embodiments, the pharmaceutical composition is configured as an oral dosage form selected from a powder, tablet, lozenge, chewing gum, a pill, a capsule, a microcapsule, a caplet, an orally disintegrating tablet, an osmotic controlled-release oral delivery system, or any combination thereof.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients (excipients). Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 0.01 to 800 mg, preferably about 0.01 mg to 400 mg, about or 3 mg to about 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions that can be used include polymeric substances and waxes. The active ingredient can also be in micro-encapsulated form, if appropriate, with one or more of the above-described excipients.

Liquid dosage forms for oral administration of compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3, or 4, part administrations. If appropriate, depending on individual behavior, it may be necessary to deviate upward or downward from the daily dose indicated.

In some embodiments, the pharmaceutical compositions disclosed herein may further comprise n-acetyl cysteine, ampelopsin, Withania somifera/ashwaganda, L-cystine, S-acetyl glutathione, molybdenum, iron, zinc, an iron chelator (Curcumin/Quercetin/IP6), L-ascorbic acid, L-threonine or any combination thereof. In some embodiments, the iron chelator comprises curcumin, quercetin, inositol hexakisphosphate (IP6), or any combination thereof. In some embodiments, the pharmaceutically acceptable carrier is water. In some embodiments, the pharmaceutical composition further comprises a taste masking agent, an odor masking agent, or a combination thereof.

In some embodiments, the therapeutically effective amount of 4-methylpyrazole is an amount between about 0.1 and about 200 mg/kg. In some embodiments, the therapeutically effective amount of 4-methylpyrazole is an amount between about 10 and about 20 mg/kg. In yet other embodiments, the therapeutically effective amount of 4-methylpyrazole is an amount resulting in a plasma concentration of about 0.01 μmol/L to about 10 μmol/L .In yet other embodiments, the therapeutically effective amount of 4-methylpyrazole is an amount resulting in a plasma concentration of about 0.1 μmol/L.

Embodiments herein are also directed to pharmaceutical compositions comprising a therapeutically effective amount of 4-methylpyrazole or pharmaceutically acceptable salts, hydrates, polymorphs, or solvates thereof, and a pharmaceutically acceptable carrier; wherein the pharmaceutical composition is configured for transdermal administration.

In some embodiments the pharmaceutical composition may further comprise n-acetyl cysteine, ampelopsin, Withania somifera/ashwaganda, L-cystine, S-acetyl glutathione, molybdenum, iron, zinc, an iron Chelator (Curcumin/Quercetin/IP6), L-ascorbic acid, L-threanine or any combination thereof. In some embodiments, the iron chelator comprises curcumin, quercetin, inositol hexakisphosphate, or any combination thereof.

Also described herein are methods of treating and/or preventing an alcohol-induced hypersensitivity reaction in a subject comprising administering a pharmaceutical composition described herein. In some embodiments, the alcohol-induced hypersensitivity reaction is selected from flushing, elevated heart rate, palpitations, hypotension, nausea, dizziness, headache, vomiting, diarrhea, upset stomach, ataxia, confused consciousness, urticarial, systemic dermatitis, allergic rhinitis, bronchoconstriction, exacerbation of asthmatic bronchoconstriction, cardiovascular collapse, allergic conjunctivitis, atopic dermatitis, eosinophilic esophagitis, anaphylaxis, chronic bronchitis and chronic obstructive pulmonary disease (COPD) and any combination thereof. In some embodiments, alcohol flushing is characterized by facial redness, increased skin temperature, elevated heart rate, decreased diastolic blood pressure or any combination thereof. In some embodiments, the pharmaceutical composition is administered before the subject consumes alcohol.

In some embodiments, the pharmaceutical composition is administered about sixty minutes to about fifteen minutes before the subject consumes alcohol. In some embodiments, the pharmaceutical composition is administered concurrently with the subject's consumption of alcohol or after the subject has consumed alcohol.

Also disclosed are methods of eliminating acetaldehyde formed from alcohol in a subject comprising administering a pharmaceutical composition described herein.

Also described herein are methods of reducing and/or eliminating acetaldehyde formed from alcohol in the oral cavity, esophagus, stomach, large intestine, or a combination thereof, in a subject, comprising administering a pharmaceutical composition described herein. Studies have shown that acetaldehyde is most abundant in the these compartments rather than in the blood stream immediately after consumption (Salaspuro M P, Acetaldehyde, microbes and cancer of the digestive tract. Crit Rev Clin Lab Sci 2003; 40:183-208; Salaspuro M. Acetaldehyde as a common denominator and cumulative carcinogen in digestive tract cancers. Scand J Gastroenterol 2009; 44:912-25; Salaspuro M. Acetaldehyde and gastric cancer. J Dig Dis 2011; 12:51-9).

Also described herein are methods of reducing and/or eliminating acetaldehyde blood levels in a subject, comprising administering a pharmaceutical composition described herein.

Also described herein are methods of reducing and/or eliminating acetaldehyde from the digestive tract in a subject comprising administering a pharmaceutical composition described herein.

Also described herein are methods of inhibiting mitochondrial aldehyde dehydrogenase 2 (ALDH2) in a subject in a subject comprising administering a pharmaceutical composition described herein.

Also described herein are methods for reducing a risk in a subject for a disease or disorder caused by consumption of alcohol in a subject comprising administering a pharmaceutical composition described herein. In some embodiments the disease or disorder is selected from upper aerodigestive tract cancers, digestive tract cancers or breast cancer. In some embodiments, the upper aerodigestive tract cancer comprises esophageal, oropharynx, hypopharynx, larynx, head or neck cancer. In some embodiments, the digestive tract cancer comprises stomach or colon cancer. In some embodiments, the disease or disorder comprises late-onset Alzheimer's disease, hypertension, myocardial infarction, Parkinson's disease, amyotrophic lateral sclerosis, and cerebral ischemia.

Some embodiments are directed to methods of blocking the histamine-releasing effect of acetaldehyde in a subject comprising administering a pharmaceutical composition disclosed herein.

Some embodiments are directed to methods of blocking alcohol induced acetaldehyde production in a subject comprising administering a pharmaceutical composition disclosed herein.

In some embodiments, the subject is heterozygous or homozygous for the aldehyde dehydrogenase 2 (ALDH2) allele termed Glu487lys, ALDH2*2, ALDH2*487lys, Glu504lys, or rs671.

Some embodiments further comprise testing the subject for the presence of aldehyde dehydrogenase 2 (ALDH2) allele termed Glu487lys, ALDH2*2, ALDH2*487lys, Glu504lys, or rs671.

In some embodiments, testing the subject for the presence of aldehyde dehydrogenase 2 (ALDH2) allele termed Glu487lys, ALDH2*2, ALDH2*487lys, Glu504lys, or rs671 comprises obtaining a breath sample from the subject; measuring alcohol and acetaldehyde levels in the breath sample; and determining the ratio of acetaldehyde level-to-alcohol level in the breath sample wherein a ratio of acetaldehyde level-to-alcohol level of about 23.3 or higher is indicative of the presence of aldehyde dehydrogenase 2 (ALDH2) allele termed Glu487lys, ALDH2*2, ALDH2*487lys, Glu504lys, or rs671; and administering the pharmaceutical composition according to claim 1 or claim 12 to the subject if the subject has a ratio of acetaldehyde level-to-alcohol level of about 23.3 or higher.

In some embodiments, measuring alcohol and acetaldehyde levels in the breath sample is done by semiconductor gas chromatography.

In some embodiments, the breath sample is obtained following consumption of alcohol.

In some embodiments, testing the subject for the presence of aldehyde dehydrogenase 2 (ALDH2) allele termed Glu487lys, ALDH2*2, ALDH2*487lys, Glu504lys, or rs671 comprises obtaining a biological sample from the subject; isolating genomic DNA from the biological sample; identifying the presence of the aldehyde dehydrogenase 2 (ALDH2) allele termed Glu487lys, ALDH2*2, ALDH2*487lys, Glu504lys, or rs671; and administering the pharmaceutical composition according to claim 1 or claim 12 to the subject if the aldehyde dehydrogenase 2 (ALDH2) allele termed Glu487lys, ALDH2*2, ALDH2*487lys, Glu504lys, or rs671 is present. In some embodiments, the biological sample is a swab sample. In some embodiments, the biological sample is a blood sample.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims. Although the present disclosure has been described in considerable detail with reference to certain preferred versions thereof, other versions are possible. Therefore, the spirit and scope of the application should not be limited to the description of the preferred versions described herein.

Although compositions, materials, and methods similar or equivalent to those described herein can be used in the practice or testing of the present invention, suitable preparations, methods and materials are described herein. All publications mentioned herein are incorporated by reference in their entirety. In the case of conflict, the present specification, including definitions will control. In addition, the particular embodiments discussed below are illustrative only and not intended to be limiting.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the application, in addition to those described herein, will be apparent to those skilled in the art from the foregoing description. Such modifications are also intended to fall within the scope of the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means plus or minus 10% of a given value. For example, “about 50% means in the range of” 45%-55%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A pharmaceutical composition comprising a therapeutically effective amount of 4-methylpyrazole, or pharmaceutically acceptable salts, hydrates, polymorphs, or solvates thereof, and a pharmaceutically acceptable carrier; wherein the pharmaceutical composition is configured for oral administration; and wherein the pharmaceutical composition further comprises at least one pharmaceutically-inert coating useful in masking the odor of 4-methylpyrazole.

2. The pharmaceutical composition of claim 1, wherein the at least one pharmaceutically-inert coating is selected from group consisting of a hydroxyalkyl cellulose, an anti-tackiness agent, a plasticizer; a sugar, a methacrylate copolymer, a hydroxyalkyl cellulose, and a water soluble polymer.

3. (canceled)

4. The pharmaceutical composition of claim 1, further comprising n-acetyl cysteine, ampelopsin, Withania somifera/ashwaganda, L-cystine, S-acetyl glutathione, molybdenum, iron, zinc, an iron chelator, L-ascorbic acid, L-threonine or any combination thereof.

5. (canceled)

6. The pharmaceutical composition of claim 1, wherein the pharmaceutically acceptable carrier is water.

7. The pharmaceutical composition of claim 1, further comprising a taste masking agent, an odor masking agent, or a combination thereof.

8. The pharmaceutical composition of claim 1, wherein the therapeutically effective amount of 4-methylpyrazole is an amount between about 10 mg/kg and about 20 mg/kg.

9. The pharmaceutical composition of claim 1, wherein the therapeutically effective amount of 4-methylpyrazole is an amount resulting in a plasma concentration of about 0.1 μmol/L.

10. A pharmaceutical composition comprising a therapeutically effective amount of 4-methylpyrazole or pharmaceutically acceptable salts, hydrates, polymorphs, or solvates thereof, and a pharmaceutically acceptable carrier; wherein the pharmaceutical composition is configured for transdermal administration.

11. The pharmaceutical composition of claim 10, further comprising n-acetyl cysteine, ampelopsin, Withania somifera/ashwaganda, L-cystine, S-acetyl glutathione, molybdenum, iron, zinc, an iron Chelator, L-ascorbic acid, L-threanine or any combination thereof.

12. The pharmaceutical composition of claim 11, wherein the iron chelator comprises curcumin, quercetin, inositol hexakisphosphate, or any combination thereof.

13. A method of treating and/or preventing an alcohol-induced hypersensitivity reaction in a subject comprising administering a pharmaceutical composition according to claim 1 to the subject.

14. The method of claim 13, wherein the alcohol-induced hypersensitivity reaction is selected from flushing, elevated heart rate, palpitations, hypotension, nausea, dizziness, headache, vomiting, diarrhea, upset stomach, ataxia, confused consciousness, urticarial, systemic dermatitis, allergic rhinitis, bronchoconstriction, exacerbation of asthmatic bronchoconstriction, cardiovascular collapse, allergic conjunctivitis, atopic dermatitis, eosinophilic esophagitis, anaphylaxis, chronic bronchitis and chronic obstructive pulmonary disease (COPD) and any combination thereof

15. (canceled)

16. The method according to claim 13, wherein the pharmaceutical composition is administered before the subject consumes alcohol.

17. (canceled)

18. The method according to claim 13, wherein the pharmaceutical composition is administered concurrently with the subject's consumption of alcohol or after the subject has consumed alcohol.

19-31. (canceled)

32. The method of any one of claim 13, further comprising testing the subject for the presence of aldehyde dehydrogenase 2 (ALDH2) allele termed Glu487lys, ALDH2*2, ALDH2*487lys, Glu504lys, or rs671.

33-38. (canceled)
